(19) United States
(12) Patent Application Publication
(10) Pub. No.: US 2025/0051183 A1
(43) Pub. Date: Feb. 13, 2025

(54) LITHIUM METAL OXIDE AND A PRECURSOR FOR THE SYNTHESIS THEREOF

(71) Applicant: UNIVERSITY COLLEGE DUBLIN, Dublin (IE)

(72) Inventor: Tony KEENE, Arklow, Co. Wicklow (IE)

(73) Assignee: UNIVERSITY COLLEGE DUBLIN, Dublin (IE)

(21) Appl. No.: 18/718,971

(22) PCT Filed: Dec. 12, 2022
(86) PCT No.: PCT/EP2022/085467

§ 371 Date: Jun. 12, 2024

(30) Foreign Application Priority Data
Dec. 13, 2021 (GB) 2118027.8

(51) Int. Cl. C01G 53/00; C01G 51/00; C07C 51/41; C07F 15/06; H01M 4/525

(52) U.S. Cl. CPC C01G 53/50; C01G 51/42; C07C 51/418; C07F 15/065; H01M 4/525; C01P 2002/50; C01P 2004/03; C01P 2006/40

(57) ABSTRACT

The present invention relates to a compound of Formula I, Li(ox)]2[MlmM2nM3k(OH)pFq; wherein M1, M2 and M3 are metals; and X is a halogen chosen from F, Cl and Br; and m, n and k are, independently, a number between 0 and 5, the sum of m, n and k is 5; p and q are, independently, a number between 0 and 8, and the sum of p and q is 8; and to uses thereof and methods of synthesis thereof.

### TECHNICAL FIELD

The present invention relates to a lithium metal oxide and a precursor for the synthesis of lithium metal oxide.

### BACKGROUND

Lithium ion batteries (LIBs) are widely used for energy storage in portable devices and electric vehicles with their growth predicted to rapidly increase as countries seek methods to decrease their emissions of greenhouse gases. However, they contain critical elements in higher concentrations than can be found naturally in their ores and have poor recycling rates. Therefore for their use to be sustainable, efficient recycling methods are needed.

Furthermore, LIBs cannot be disposed of in a landfill safely because they contain a flammable electrolyte and heavy metals. Additionally, many of the metals used are critical elements in low abundance such as cobalt and lithium. LIBs often contain 10-20 wt % of lithium and cobalt, which is far higher than is naturally found in their ores. 35% of lithium and 25% of cobalt produced globally is used in the LIB industry. Both elements have a low abundance in the earth's crust and so element scarcity is a grave concern.

Lithium cobalt oxide (LCO) remains the most common choice of cathode but several other cathodes have been developed in response to the scarcity and cost of cobalt. For example, LiNiO₂ is a layered transition metal oxide that is isostructural to LCO. However it has been found to be less stable than LCO because nickel and lithium ions can swap sites. The addition of cobalt to form LiNi₁₋ₓCoₓO₂ results in a cathode with greater stability when x is greater than 0.3. However, the performance is still slightly poorer than LCO so further substitutions with aluminium (LiNi_{1-x-y}CoₓAl_{y}O₂ also known as NCA) and manganese (LiNi_{0.5-x}Mn_{0.5-x}CoₓO2 also known as NMC) are used to improve the performance and are commercially used as alternative cathodes to LCO. Although research is ongoing to reduce the cobalt content in cathodes, the most popular commercial batteries still include cobalt due to superior performance.

As of 2020, the most common cathodes used in commercially available LIBs are LCO (37%), NMC (29%), LiMn₂O₄ (22%), LiNiO₂ (7%) and LiFePO₄ (5%). (Y. Bai, N. Muralidharan, Y. K. Sun, S. Passerini, M. S. Whittingham and I. Belharouak, Mater. Today, 2020, 41, 304-315.)

Known methods for recycling cobalt and other metals from batteries suffer from the limitation that they have low tolerance to impurities. Known methods also involve long reaction times, many steps, dangerous reagents and high temperatures, and many are also low yielding and are not scalable. Many methods require strong acids and other corrosive chemicals which can destroy conventional reactors.

An improved method for extracting and re-using the metal components of used batteries is therefore needed. It is also envisaged that the present disclosure can be applied to the recovery of metals from a variety of sources.

### SUMMARY

According to a first aspect of the invention, there is provided a compound of Formula I,

[Li(dicarboxylate)]₂[M¹ₘM²ₙM³ₖ(OH)ₚX_{q}];
wherein M¹, M² and M³ are metals; and
X is a halogen chosen from F, Cl, Br and I; and
m, n and k are, independently, a number between 0 and 5, the sum of m, n and k is 5;
p and q are, independently, a number between 0 and 8, and the sum of p and q is 8.

By the term “number” is meant positive whole numbers and fractions thereof.

The compound of Formula I is a precursor for the synthesis of a lithium metal oxide for use in batteries. Since battery anode and cathode materials may comprise a variety of metals in a varying ratios depending on the properties sought, so too can the compound of Formula I.

M¹, M² and M³ may be the same or different and may be independently selected from cobalt, nickel, manganese, zinc, copper and iron.

Optionally, M¹ is nickel, M² is manganese and M³ is cobalt.

Optionally, m and n are 0; k is 5; and M³ is cobalt.

Optionally, at least one of M¹, M² and M³ is selected from a metal which can form a tetracoordinate compound, such as zinc, iron or cobalt, preferably cobalt or zinc. This has been found to aid crystallization.

Each dicarboxylate, which may be the same or different, may comprise 2-8 carbon atoms.

Optionally each dicarboxylate is independently chosen from
(i) a linear saturated dicarboxylate chosen from oxalate, malonate, succinate, glutarate, adipate, pimelate, suberate, azelate, sebacate or a mixture thereof;
(ii) an unsaturated dicarboxylate chosen from maleate, fumarate, acetylenedicarboxylate, glutaconate, traumatate, muconate, glutinate, citraconate, mesaconate, itaconate, tartronate, mesoxalate, malate, tartrate, oxaloacetate, aspartate, dioxosuccinate, alpha-hydroxy glutarate, acetonedicarboxylate or alpha-ketoglutarate or a mixture thereof; or
(iii) one dicarboxylate is a linear saturated dicarboxylate selected from (i) and one dicarboxylate is an unsaturated dicarboxylate selected from (ii).

Optionally, each dicarboxylate is oxalate.

Optionally, Formula I is [Li(ox)]₂[CO₅(OH)₈] which can also be written as [Li(C₂O₄)]₂[Co₅(OH)₈]; or Formula I is [Li(ox)]₂[Ni_{5/3}Mn_{5/3}CO_{5/3}(OH)₈], which can also be written as [Li(C₂O₄)]₂[Ni_{5/3}Mn_{5/3}CO_{5/3}(OH)₈], wherein 5/3 represents five thirds. It should be understood that “ox” represents “oxalate”.

According to a second aspect of the invention, there is provided a method for synthesizing the compound of Formula 1 of the first aspect of the invention, the method comprising:
a first step comprising heating a first reaction mixture comprising a source of the metal or metals, M¹, M² and M³; a source of a dicarboxylate ions such as a dicarboxylic acid, a dicarboxylate salt or a mixture thereof; lithium ions; and a base at a first reaction temperature for a first reaction time.

Optionally the source of the metal or metals M¹, M² and M³, and the source of the dicarboxylate ions is provided by a metal dicarboxylate.

The method can be performed with a wide variety of starting materials, provided the ions which form the compound of Formula I are present in the first reaction mixture. The process is particularly attractive for recycling as it works with a variety of metal sources with a variety of metal oxidation states. Mixtures of starting materials and impurities are tolerated. For example, the metals M¹, M² and M³ may be metals obtained from used batteries, metal scrap waste such as used vehicles and their components, electronic waste, waste products from metal catalyzed reactions, domestic and industrial waste, effluent from mining operations and metals obtained from domestic, agricultural and industrial wastewater.

The method is high yielding, with yields of over 95% being attained. Most advantageously, the compound of Formula I is formed as a crystalline solid with a particle size of 5 to 2000 μm. This makes it easily recoverable in a pure form from the remaining reaction mixture by filtration, centrifugation, sedimentation or evaporation. It is surprising that the compound of Formula I is recoverable as a crystalline solid as lithium compounds are usually soluble. Crystalline compounds are also safer to handle than the fine powders produced by many of the existing methods of metals recovery.

The first heating step may be carried out in a sealed reactor such as a pressure reaction vessel, such as a solvothermal reactor, or an acid digestion vessel. The reagents are mixed with solvent and the reactor is sealed and heated. In such reactors, reactions can also be carried out at temperatures above the boiling point of the solvent, allowing the possibility of modes of reactivity unattainable below the boiling point of common solvents. The resulting high temperatures inside the reactor can allow the dissolution and reaction of components which do not dissolve at room temperature, or which dissolve at low concentration only at room temperature. Such conditions make higher concentrations of starting materials viable. Furthermore, it has been surprisingly found that performing the reaction in a sealed reactor under pressure helps the formation of crystals of the compound of Formula I of the first aspect of the invention.

Optionally, one or more of M¹, M² and M³ are provided as cations, for example as a salt. Any salt which provides the requisite ions may be used provided it dissolves under the reaction conditions. When a sealed reactor is used, many salts become soluble which are not soluble at room temperature and atmospheric pressure.

Optionally, the, or each, M¹, M² and M³ salt is selected from a cobalt (II) or (III) salt, a manganese (II) or (III) salt or a nickel (II) or (III) salt, or a mixture thereof. It has been found that the metal (III) ions reduce to metal (II) ions in the reactor when the solvent is water. It is proposed that the reaction conditions change the dissociation constant of water so that water becomes a reducing agent. This is particularly beneficial for a recycling process where the starting materials may be a mixture of compounds.

Optionally, the, or each, M¹, M² and M³ salt is soluble in water under self-generated pressure, at a temperature of 100° C. up to 300° C. and a concentration of 0.5M up to 10M.

Optionally, the salt is selected from a halide, such as chloride, bromide, or iodide; a carboxylate such as an alkylenedioate optionally selected from oxalate, acetate, citrate, formate; an oxoanion such as fluorosilicate, nitrate, nitrite, or sulfate salt, acetate, triflate, or a mixture thereof. The method tolerates a variety of reagents and mixtures of reagents provided that the ions which constitute the compound of Formula I are present.

Optionally, the source of metal or metals is selected from cobalt chloride or cobalt oxalate.

Optionally, the source of metal or metals and the source of dicarboxylate anions are collectively selected from cobalt oxalate, manganese oxalate, nickel oxalate, cobalt oxalate or a mixture of two or more thereof, wherein the source of metal or metals and the source of dicarboxylate anions is anhydrous or a hydrate, such as monohydrate, dihydrate or trihydrate. When the dicarboxylate in the compound of Formula I is oxalate, the compound of Formula I has been found to be crystalline and stable.

Optionally, the source of dicarboxylate anions is a dicarboxylic acid selected from a dicarboxylic acid such as oxalic acid or a hydrate thereof, or a dicarboxylate salt such as an oxalate salt. A mixture of sources of oxalate can also be used.

Optionally, the lithium ion is selected from a lithium salt or a lithium base or a mixture thereof. The method tolerates mixtures of sources. For example, some of the lithium ions may come from recovered battery material and some may be added prior to the reaction.

Optionally, the anion of the lithium cation is a halide such as chloride, bromide or iodide; a carboxylate such as oxalate, acetate or benzoate; a halite such as bromate or chlorate; or fluorosilicate, formate, molybdate, nitrate, nitrite, perchlorate, permanganate, selenide, selenite, sulfate, thiocyanate.

Optionally, the lithium base is lithium hydroxide, optionally in anhydrous or monohydrate form. A lithium base can perform the function of the lithium and the base in the first reaction mixture.

Optionally, the halogen is fluorine and the source of fluorine is lithium fluoride. The fluoride ion resembles the hydroxide ion. Both carry a single negative charge. The ionic radius of the fluoride ion is 119 μm while the effective ionic radius of the hydroxide ion is 110 μm. Fluoride ions are also soluble in protic solvents such as water, like hydroxide ions. The two ions are known to participate in similar reactions and occupy the same position in crystal lattices. For example, when fluoride containing toothpaste is applied to teeth, fluoride readily displaces hydroxide in the crystal lattice of hydroxyapatite to form fluorohydroxyapatite. Zhijuan Liu et al. in J. Mater. Chem. A, 2019, 7, 14483-14488, DOI: 10.1039/C9TA03882E, “Modulating the electronic structure of ultrathin layered double hydroxide nanosheets with fluorine: an efficient electrocatalyst for the oxygen evolution reaction” disclose another ordered solid structure in which fluorine can replace hydroxide.

Optionally, the method further comprises the step of recovering the lithium ions from batteries.

Optionally, the method further comprises the step of recovering, or extracting, the source of the metal or metals from batteries.

Optionally, the source of metal or metals M¹, M² and M³ is recovered, or extracted, by mixing the contents of a battery with an acid, such as a weak acid, such as oxalic acid, malonic acid, succinic acid, glutaric acid or adipic acid in water for at least 30 minutes, optionally 1 to 8 hours, optionally about 4 hours, thereby forming a metal dicarboxylate.

Optionally the source of metal or metals M¹, M² and M³ is extracted by converting lithium metal oxide into a metal dicarboxylate by refluxing the lithium metal oxide in water and a dicarboxylic acid, optionally oxalic acid, for 1 to 8 hours, optionally for approximately 4 hours. The lithium metal oxide is optionally derived from used batteries and is optionally LCO or NMC. Approximately three molar equivalents of dicarboxylic acid may be used for the extraction. The metal dicarboxylate may be recovered by filtration.

Optionally, the base is selected from lithium hydroxide, sodium hydroxide, potassium hydroxide, or a mixture thereof.

Optionally, the source of dicarboxylate anions is selected from an alkylenedioic acid or a salt or anion thereof such as oxalic acid, malonic acid, succinic acid, glutaric acid or adipic acid or a salt or anion thereof, or a mixture thereof.

Optionally, the dicarboxylic acid or a salt or anion thereof is selected from oxalic acid or an oxalate salt such as a metal oxalate such as lithium oxalate, cobalt oxalate, nickel oxalate or manganese oxalate.

Optionally, the first reaction time is between 1 hour and 100 hours; optionally between 1 hour and 50 hours; further optionally between 2 hours and 50 hours; still further optionally between 3 hours and 18 hours, or approximately 4 hours.

Optionally, the first reaction temperature is in the range from 150° C. to 400° C.; further optionally from 200° C. to 300° C.; still further optionally approximately 200° C. to 230° C. 200° C. to 230° C. is an optimal balance as lower temperatures slow the reaction but higher temperatures require more energy and may cause rust to form. However, the reaction has also been found to proceed at temperatures from 90° C. up to 100° C., such as 95° C. This temperature range does not generate as much pressure but uses less energy.

Optionally, the method further comprises the step of cooling the first reaction mixture. Cooling may be gradual to favour the formation of larger crystals.

Optionally, the method further comprises the step of collecting the compound of Formula I, optionally by filtration, centrifugation, sedimentation or evaporation. Further processes such as sieving or screening may be carried out.

Optionally, halogen anions are not intentionally added to the source of the metal or metals, the source of dicarboxylate anions, the lithium ions, and the base. This can reduce the wear on the reactor, especially if the reactor is made from Hastelloy®. It may also allow the reaction to be carried out in a reactor made from stainless steel.

Optionally, impurities are present during the first heating step. As mentioned above, the method is very tolerant to impurities. The compound of Formula I is crystalline which allows the impurities present in the starting materials and any side products or by-products to be separated from the compound of Formula I after the reaction.

Optionally, the impurities comprise aluminium such as aluminium (0) metal or aluminium oxide, or copper, such as copper (0) metal or copper oxalates or copper oxides, iron such as iron (0) metal, iron oxides or iron oxalates or mixtures thereof.

Optionally, the first heating step is carried out in a protic solvent such as water. Water is particularly preferred as it dissolves most salts and has a relatively high boiling point, is readily available and it is considered environmentally friendly. Furthermore, it is envisaged that the crude starting materials may be provided in an aqueous solution for example, after dissolving batteries in acid, or as wastewater. When the reaction is carried out in a sealed reactor, the water can become a reductant which can ensure that the metal ions are in the correct oxidation state for the formation of the compound of Formula I. For example, Ni(III) may be reduced to Ni(II) under the reaction conditions.

Optionally, the source of the metal or metals and the source of dicarboxylate anions collectively comprise cobalt oxalate, and the lithium ions and the base collectively comprise lithium hydroxide. These comprise an atom efficient source of the ions required to form the compound of Formula I of the first aspect of the invention.

Optionally, the source of the metal or metals collectively comprise cobalt chloride, the source of dicarboxylate anions is oxalic acid (anhydrous or dihydrate), the source of the lithium ions is lithium chloride, lithium bromide or lithium hydroxide and the base is either lithium hydroxide or sodium hydroxide. These comprise an alternative atom efficient source of the ions required to form the compound of Formula I.

Optionally, in the first heating step, the concentration of the metal or metals combined is at least 10 mmol/L. A wide range of concentrations is tolerated by the method. However, reaction times are aided by increased concentration. Furthermore, sealed reactors are typically smaller than standard reactors.

Optionally, the molar ratio of metal ions to lithium ions in the first reaction mixture before the first heating step is in the range of 1:10 to 5:1 optionally, in the range of 1:1 to 1:3; further optionally wherein the molar ratio of metal ions to lithium ions is 1:2. Increased lithium improves reaction times. Lithium is more abundant and more environmentally benign than most of the metals which the method aims to recover, so an excess of lithium is a preferable way to increase reaction time.

Also provided is a method for synthesizing lithium metal oxide, the method comprising:
forming the compound of Formula I; and further comprising a second heating step comprising heating the of Formula I at a second reaction temperature for a second reaction time to form the lithium metal oxide.

The formed lithium metal oxide may be used to manufacture new batteries.

An example of the reaction where the only metal is cobalt is as follows:

[Li(ox)]₂[Co₅(OH)₈]→2LiCoO₂+Co₃O₄+4H₂O+4CO₂

The progress of the reaction can be monitored by mass loss. For example, in the above example, the predicted mass loss is 29%,

Optionally, the second reaction time is between 1 hour and 100 hours, optionally between 5 and 20 hours, further optionally between 7 and 12 hours, still further optionally approximately 8 hours. Alternatively, the reaction can be monitored and stopped when the predicted mass loss is observed.

Optionally, the second reaction temperature in the range from 200° C. to 900° C., optionally from 250° C. to 450° C., further optionally form 250° C. to 400° C., still further optionally from 250° C. to 350° C., even still further optionally from 275° C. to 325° C., optionally from 275° C. to 400° C., 300° C. to 400° C., or approximately 300° C. As will be discussed in relation to the drawings, the temperatures at which decomposition to lithium metal oxide occurs for [Li(ox)]₂[Co₅(OH)₈] and [Li(ox)]₂[Ni_{5/3}Mn_{5/3}Co_{5/3}(OH)₈] have been found to be different. Therefore, the second reaction temperature depends on the exact composition of [Li(dicarboxylate)]₂[M¹ₘM²ₙM³ₖ(OH)ₚX_{q}] and should be chosen accordingly. In other words, the second reaction temperature is the temperature at which the [Li(dicarboxylate)]₂[M¹ₘM²ₙM³ₖ(OH)ₚX_{q}] starts to decompose to lithium metal oxide.

Optionally, the method includes determining the second reaction temperature, optionally via thermogravimetric analysis, optionally followed by PXRD analysis to confirm the identity of the formed product, or by subjecting the compound of Formula I to increasing temperatures and observing at which temperature it begins to decompose to lithium metal oxide by other methods.

Optionally, the lithium metal oxide is lithium nickel manganese cobalt oxide of the formula Li(Ni/Mn/Co)O₂ wherein the sum value of Ni, Mn and Co is 1, the value of each metal is between 0 and 1, or wherein the lithium metal oxide is LiCoO₂, or LiNiO₂ or LiMn₂O, or LMO₂ where M=Ni/Co/Mn in the ratio of the PM-2 parent compound

Optionally, the second heating step is carried out in an open vessel, such as a kiln, a cement roasting kiln or a furnace or a laboratory flask such as a roundbottom flask.

Optionally, the compound of Formula I of the first aspect of the invention is crystalline. This makes handling of the material simpler and ensures greater purity. When the crystalline compound of Formula I is heated, the formed lithium metal oxide is also formed as a crystalline solid. Since the compound of Formula I is isolatable as a pure crystalline solid, the subsequent reaction to form the lithium metal oxide is very clean.

Optionally, the compound of Formula I has a particle size of about 5 to 2000 μm. It has been found that the crystal size of the formed lithium metal oxide is smaller than the crystal size of the compound of Formula I.

Optionally, the formed lithium metal oxide is crystalline and has a particle size of 500 nm up to 500 μm.

### BRIEF DESCRIPTION OF DRAWINGS

FIG. 1a shows a single crystal of [Li(ox)]₂[Co₅(OH)₈] synthesized via Method 1. FIG. 1b depicts the stacking of layers in [Li(ox)]₂[Co₅(OH)₈].

FIG. 2 shows the powder X-ray diffraction pattern was measured for the same [Li(ox)]₂[Co₅(OH)₈] as for FIG. 1a. The top line is processed for reading, and the middle one still has to be corrected so that all the signals are “positive”.

FIG. 3 shows the thermogravimetric analysis of [Li(ox)]₂[Co₅(OH)₈] leading to formation of LiCoO₂ and Co₃O₄.

FIG. 4A shows the Scanning Electron Microscopy image of [Li(ox)]₂[Co₅(OH)₈] prior to decomposition; FIG. 4B shows the resulting compound after decomposition of [Li(ox)]₂[Co₅(OH)₈].

FIG. 5 shows the asymmetric unit and selected symmetry equivalents of [Li(C₂O₄)]₂[M₅(OH)₈](where M=1:1:1 Ni:Mn:Co) from single crystal data collection. Occupancy of each metal site fixed by electronic preference of metal(II) ions within the statistical mixture. Symmetry operators as for FIG. 1.

FIG. 6 shows powder X-ray diffraction plot of [Li(C₂O₄)]₂[M₅(OH)₈] where M=1:1:1 Ni:Mn:Co with le Bail profile fit to confirm phase identity.

FIG. 7 shows the stacked PXRD plots of different NMC ratios. Additional peaks are from lithium hydrogenoxalate monohydrate.

FIG. 8 shows the thermogravimetric analysis (TGA, black trace) plot of [Li(C₂O₄)]₂[M₅(OH)₈](where M=1:1:1 Ni:Mn:Co) in air.

### DESCRIPTION OF EMBODIMENTS

### Abbreviations

FT-IR—Fourier-transform infrared spectroscopy
LCO—Lithium cobalt oxide
LIBs—Lithium-ion batteries
NMC—Lithium nickel manganese cobalt oxide
PM-1—Precursor material for LCO regeneration, [Li(ox)]₂[Co₅(OH)₈]
PM-2—Precursor material for NMC regeneration, [Li(ox)]₂[M₅(OH)₈], wherein M=
Ni:Mn:Co in varying ratios
PXRD—Powder X-ray diffraction

### Synthesis of PM-1, [Li(ox)]₂[Co₅(OH)₈]

### Method 1 for Synthesis of PM-1

PM-1 was attained by adding COCl₂·6H₂O (1 mmol) 237 mg, LiOH·H₂O (3.408 mmol) 143.7 mg, LiBr (3 mmol) 261 mg and C₂H₂O⁴·H₂O (1 mmol) 126 mg to a 23 mL Teflon lined bomb reactor with 10 mL of deionised water. This vessel was heated at 230° C. for 4 hours. The bomb was left to cool, and the contents filtered.

The yield of [Li(ox)]₂[Co₅(OH)₈] was over 95%.

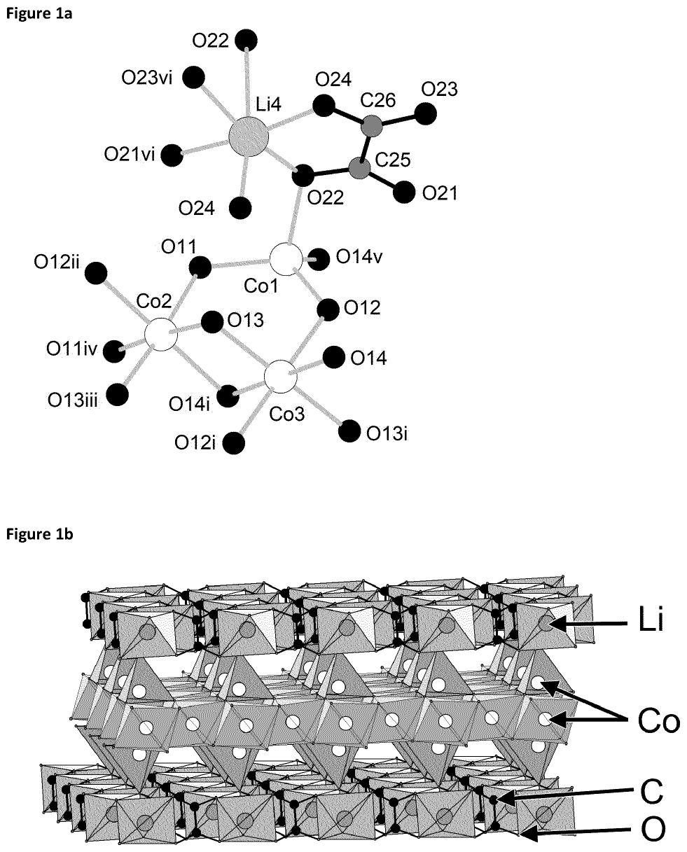

Single crystal X-ray diffraction and powder X-Ray diffraction analysis were carried out to confirm the structure. Thermogravimetric analysis was carried out to monitor conversion to lithium cobalt oxide LiCoO₂, see FIGS. 1-4.

### Conversion of LCO to PM-1

### Method 2

### Step A: Conversion of LCO to Cobalt Oxalate

LCO (294.1 mg, 3 mmol), oxalic acid dihydrate (1.1345 g, 9 mmol) and water (30 mL) were added to a round bottom flask (50 mL) and refluxed in a glycerol bath at 100° C. for 4 hours until the reaction mixture was entirely pink. The product was filtered and the colourless filtrate was collected. The product was washed with water and acetone. Yield>95%.

### Step B: Conversion of Cobalt Oxalate to PM-1

Cobalt oxalate dihydrate from part A (182.5 mg, 1 mmol) and lithium hydroxide solution (1 M) (2.0 mL, 2 mmol) were placed in a sealed vessel with water (8 mL). The vessel was placed in an oven at 200° C. for 14 hours. A green powder was formed in a colourless liquid.

The yield of PM-1, [Li(ox)]₂[Co₅(OH)₈] was >95%.

Extraction of Cobalt Oxalate from LCO with Impurities Found in Spent Batteries
Extraction of Cobalt Oxalate from LCO with Al Foil Present

LCO (195.6 mg, 2 mmol), oxalic acid dihydrate (0.7452 g, 6 mmol), aluminium foil (97.4 mg, 3.6 mmol) and water (20 mL) were added to a round bottom flask (50 mL) and refluxed in a glycerol bath at 100° C. for 4 hours until the reaction mixture was beige. The product was filtered and the colourless filtrate was collected. The product was washed with water.

Extraction of Cobalt Oxalate from LCO with Cu Turnings Present

LCO (195.4 mg, 2 mmol), oxalic acid dihydrate (0.7442 g, 6 mmol), copper turnings (122.0 mg, 1.9 mmol) and water (20 mL) were added to a round bottom flask (50 mL) and refluxed in a glycerol bath at 100° C. for 4 hours until the reaction mixture was pink. Copper turnings were still present in the flask and the solution was blue in colour. The product was filtered and the filtrate was collected. The product was washed with water.

Extraction of Nickel Manganese Cobalt Oxalate from NMC

NMC (192.5 mg, 2 mmol), oxalic acid dihydrate (0.7448 g, 6 mmol) and water (20 mL) were added to a round bottom flask (50 mL) and refluxed in a glycerol bath at 100° C. for 4 hours until the reaction mixture was brown. The product was filtered and the colourless filtrate was collected. The product was washed with water and acetone.

### Synthesis of PM-1 with Impurities Found in Spent Batteries

### Synthesis of PM-1 with Cu Turnings Present

Cobalt oxalate dihydrate (183.8 mg, 1 mmol), lithium hydroxide solution (1 M) (2.0 mL, 2 mmol) and copper turnings (61.9 mg, 1 mmol) were placed in a sealed vessel with water (8 mL). The vessel was placed in an oven at 200° C. for 14 hours. A green powder with copper turnings still present was filtered from a colourless solution.

### Synthesis of PM-1 with Copper Oxalate Dihydrate Present

Cobalt oxalate dihydrate (92.4 mg, 0.5 mmol), copper oxalate dihydrate (76.4 mg, 0.5 mmol) and lithium hydroxide solution (1 M) (2.0 mL, 2 mmol) and were placed in a sealed vessel with water (8 mL). The vessel was placed in an oven at 200° C. for 14 hours. A shiny black powder with a few green crystals was filtered from a blue solution.

### Synthesis of Metal Oxalates

Cobalt oxalate dihydrate, nickel oxalate dihydrate and zinc oxalate dihydrate were already available for use as reagents but iron oxalate dihydrate, manganese oxalate dihydrate and cobalt nickel manganese oxalate had to be synthesised prior to use.

### Synthesis of Iron Oxalate Dihydrate

Oxalic acid dihydrate (2.5064 g, 19.88 mmol) and concentrated sulfuric acid (0.3 mL) were added to water (50 mL) and heated to 55° C. while being stirred till the oxalic acid dissolved. Iron (II) ammonium sulfate (5.0011 g, 12.75 mmol) was added and a yellow precipitate formed instantaneously. The precipitate was filtered for use in further reactions.

### Synthesis of Manganese Oxalate Dihydrate

Oxalic acid dihydrate (2.5133 g, 19.94 mmol) and concentrated sulfuric acid (0.3 mL) were added to water (25 mL) and heated to 55° C. while being stirred till the oxalic acid dissolved. Manganese (II) acetate tetrahydrate (3.1261 g, 12.75 mmol) was added and a white precipitate formed instantaneously. The precipitate was filtered for use in further reactions.

### Synthesis of Cobalt Nickel Manganese Oxalate

Oxalic acid dihydrate (14.4928 g, 0.115 mol) and concentrated sulfuric acid (1 mL) were added to water (75 mL) and heated to 55° C. while being stirred till the oxalic acid dissolved. Manganese (II) acetate tetrahydrate (9.0693 g, 0.037 mol), nickel (II) acetate tetrahydrate (9.2052 g, 0.037 mol) and cobalt (II) chloride hexahydrate (8.8029 g, 0.037 mol) was added and a fine purple precipitate formed instantaneously. The precipitate was filtered for use in further reactions.

### Synthesis of PM-2, [Li(ox)]₂[M₅(OH)₈] Wherein M=Ni:Mn:Co in Varying Ratios

The syntheses were carried out in the same way as above example wherein M=1:1:1: Ni:Mn:Co, with only the relative amounts of metal ion sources changed. For example, instead of using only CoCl₂·6H₂O, as per “Method 1 for synthesis of PM-1” above, a mixture of cobalt chloride, nickel chloride and manganese chloride may be used. Alternatively, instead of using cobalt oxalate, as per “Method 2 for synthesis of PM-1” above, cobalt nickel manganese oxalate may be used, for example. Again, other sources of metal ions, oxalate ions and lithium ions are tolerated by the method. Impurities, for example iron ions and iron oxide, are also tolerated.

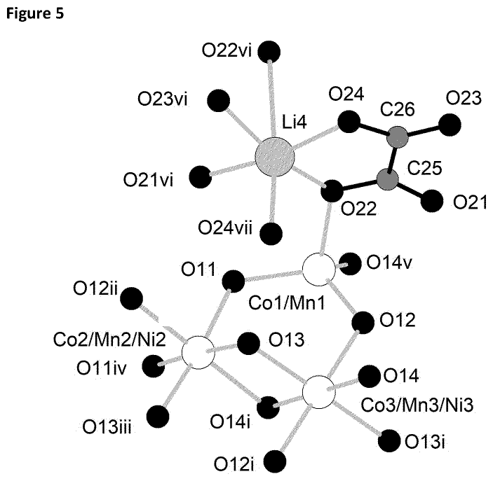

Single crystal X-ray diffraction and powder X-Ray diffraction analysis were carried out to confirm the structure. Thermogravimetric analysis was carried out to monitor conversion to lithium metal oxide, LiMO₂, see FIGS. 5-8.

### Synthesis of Nickel and Cobalt Based Precursor Material

Nickel oxalate dihydrate (91.3 mg, 0.5 mmol), cobalt oxalate dihydrate (90.6 mg, 0.5 mmol) and lithium hydroxide monohydrate (83.3 mg, 2 mmol) were placed in a sealed vessel with water (10 mL). The vessel was placed in an oven at 200° C. for 14 hours. A green powder was filtered.

### Synthesis of Manganese, Nickel and Cobalt Based Precursor Material

CoCl₂·6H₂O (2 molar solution in water, 166 μl, 0.33 mmol), NiCl₂·6H₂O (1 molar solution in water, 333 μl, 0.33 mmol), MnCl₂·4H₂O (1 molar solution in water, 333 μl, 0.33 mmol), LiOH·H₂O (3 molar solution in water, 2.0 ml, 6 mmol), LiCl (5 molar solution in water, 2.4 ml, 12 mmol) C₂H₂O₄·2H₂O (1 molar solution in water, 2.5 ml, 2.5 mmol) were added to a 23 mL Teflon lined bomb reactor and made up to 10 mL with deionised water. This vessel was heated at 230° C. for 12 hours. The bomb was left to cool, and the contents filtered. The yield of [Li(ox)]₂[Mn/Ni/Co₅(OH)₈] was over 95%.

### Characterisation of the Formed Precursor Materials

Single crystal X-ray diffraction data were collected in house on a Rigaku Oxford Diffraction SuperNova A diffractometer fitted with an Atlas detector with Cu-Kα (1.54184 Å) and Mo-Kα (0.71073 Å).

Referring again to FIG. 1a which shows the results of single crystal X-Ray diffraction of [Li(ox)]₂[Co₅(OH)₈](synthesized by Method 1), it can be seen that the compound crystallises in the triclinic space group P-1 (space group no. 2). The asymmetric unit is comprised of 3 cobalt metal centres, 4 hydroxides, 1 lithium metal centre and 1 oxalate. Two of the cobalt hydroxide centres (Co2; Co3) have near octahedral geometry and are bridged to one another through hydroxides O13 and O14i. The third cobalt centre (Co₁) has near tetrahedral geometry and is coordinated to Co₂ and Co₃ through hydroxyl oxygens 011 and 012 respectively. The lithium metal centre (Li4) is coordinated by 5 oxygen atoms, from the oxalate ligand (O22 and O24) and 3 symmetry generated oxygen atoms from neighbouring oxalate moieties (O21vi, O23vi and O24vii). Li4 is bridged to Co₁ through O22. Symmetry equivalents: i) 1-x, 1-y, 1-z; ii) 1+x, y, z; iii) 2-x, 1-y, 1-z; iv) 2-x, 2-y, 1-z; v) x, 1-y, z; vi) 1+x, y, z.

FIG. 1b depicts the stacking of the layers of [Li(ox)]₂[Co₅(OH)₈] with the [Li(ox)]⁻¹ layer shown in the top and bottom layers and [Co₅(OH)₈]²⁺ represented by the middle layer. The cobalt can be octahedrally coordinated or tetrahedrally coordinated where systematic depletions occur. One of the reasons this recycling method is successful is due to [Li(ox)]₂[Co₅(OH)₈] having a layered structure. LCO also contains a layered structure, which allows calcination to be carried out at comparatively low temperatures. This makes the process energy efficient and simple.

The bond lengths and angles of the above crystal structure are given in the below Table 1.

TABLE 1

| M—L | d/Å | | L—M—L | </° | | L—M—L | </° | |
|---|---|---|---|---|---|---|---|---|
| Co1—O14v | 1.935 | (15) | O14v—Co1—O12 | 114.5 | (6) | O12—Co3—O14i | 90.8 | (5) |
| Co1—O12 | 1.953 | (14) | O14v—Co1—O11 | 112.7 | (6) | O12—Co3—O14 | 89.2 | (5) |
| Co1—O11 | 1.957 | (14) | O14v—Co1—O22 | 108.3 | (7) | O12i—Co3—O14 | 90.8 | (5) |
| Co1—O22 | 2.116 | (19) | O12—Co1—O11 | 115.0 | (6) | O12i—Co3—O12 | 180 | |
| Co2—O14i | 2.130 | (13) | O12—Co1—O22 | 99.9 | (7) | O13—Co3—O14i | 84.5 | (5) |
| Co2—O12ii | 2.123 | (13) | O11—Co1—O22 | 104.9 | (6) | O13i—Co3—O14i | 95.5 | (5) |
| Co2—O13 | 2.057 | (14) | O12ii—Co2—O14i | 176.8 | (5) | O13i—Co3—O14 | 84.5 | (5) |
| Co2—O13iii | 2.093 | (13) | O13—Co2—O14i | 84.7 | (5) | O13—Co3—O14 | 95.5 | (5) |
| Co2—O11iv | 2.081 | (14) | O13iii—Co2—O14i | 93.8 | (5) | O13i—Co3—O12 | 85.0 | (5) |
| Co2—O11 | 2.122 | (13) | O13iii—Co2—O12ii | 84.5 | (5) | O13i—Co3—O12i | 95.0 | (5) |
| Co3—O14 | 2.147 | (14) | O13—Co2—O12ii | 92.3 | (5) | O13—Co3—O12 | 95.0 | (5) |
| Co3—O14i | 2.147 | (14) | O13—Co2—O13iii | 82.2 | (6) | O13—Co3—O12i | 85.0 | (5) |
| Co3—O12 | 2.147 | (14) | O13—Co2—O11iv | 175.3 | (5) | O13—Co3—O13i | 180 | |
| Co3—O12i | 2.147 | (14) | O13—Co2—O11 | 97.4 | (5) | O23vi—Li4—O24vii | 91. | (2) |
| Co3—O13i | 2.048 | (13) | O13iii—Co2—O11 | 174.8 | (5) | O22—Li4—O23vi | 167. | (3) |
| Co3—O13 | 2.048 | (13) | O11—Co2—O14i | 91.3 | (5) | O22—Li4—O24vii | 102. | (2) |
| Li4—O21vi | 2.04 | (6) | O11iv—Co2—O14i | 90.7 | (5) | O22—Li4—O24 | 83. | (2) |
| Li4—O22 | 1.96 | (6) | O11iv—Co2—O12ii | 92.3 | (5) | O22—Li4—O21vi | 98. | (3) |
| Li4—O23vi | 2.12 | (6) | O11—Co2—O12ii | 90.4 | (5) | O24—Li4—O23vi | 95. | (2) |
| Li4—O24 | 2.01 | (6) | O11iv—Co2—O13iii | 98.1 | (5) | O24—Li4—O24vii | 91. | (2) |
| Li4—O24vii | 2.34 | (6) | O11iv—Co2—O11 | 82.7 | (5) | O24—Li4—O21vi | 170. | (3) |

| O14—Co3—O14i | 180 | | O21vi—Li4—O23vi | 83. | (2) |
|---|---|---|---|---|---|
| O12i—Co3—O14i | 89.2 | (5) | O21vi—Li4—O24vii | 98. | (2) |

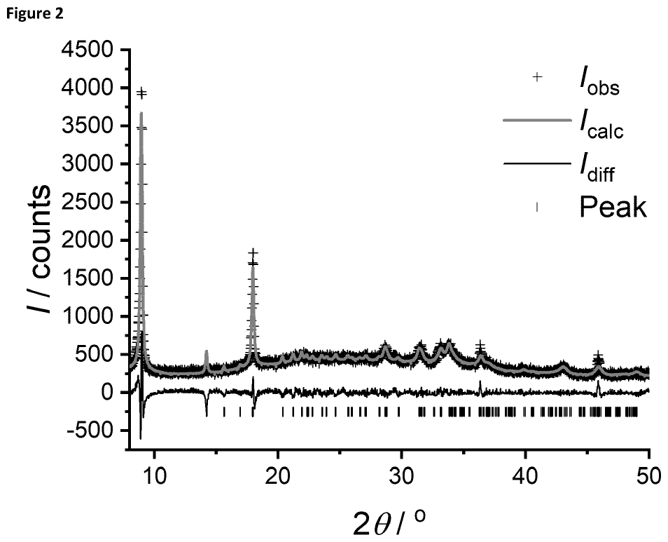

Referring again to FIG. 2, the powder X-ray diffraction pattern was measured for [Li(ox)]₂[Co₅(OH)₈] and Le Bail profile fitted using the cell parameters obtained from the single crystal X-ray data collection. Powder X-ray diffraction (Cu-Kα, λ=1.5406) plot with Le Bail profile fit for [Li(ox)]₂[Co₅(OH)₈]. Pbcn, a=5.3525 (5) Å, b=6.3448 (5) Å, c=10.1774 (16) Å, α=98.920°, β=100.084 (7) °, γ=90.373 (8) °, V=335.9 (7) (1.4), Rₚ=8.85, R_{wp}=12.00, Rₑₓₚ=5.48. Powder X-Ray data were collected on a Siemens D500 diffractometer with a Cu-Kα source. Le Bail profile fits on powder X-ray data were performed in Rietica to ensure phase identity and sample purity.

Elemental analysis was performed on an Exeter Analytical CE 440 elemental analyser. Expected for PM-1 (%): C:7.74, H:1.30; Found: C7.44, H:1.08.

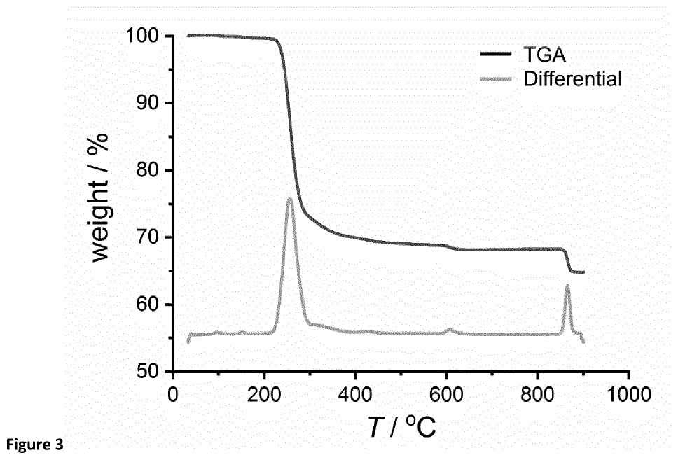

Referring again to FIG. 3, there is shown the thermogravimetric analysis of the [Li(ox)]₂[Co₅(OH)₈]. TGA/MS was measured with TGA Q500 thermogravimetric balance with Evolved Gas Analysis (EGA) furnace. Mass Spectrometer Edwards HPR20. Initial isotherm for 30 minutes at room temperature, followed by heating under in air flow to 900° C., at a ramp rate of 5° C./min, then a final 30-minute isotherm at final temperature. Sample size was 2.143 mg of dry solid.

Shown is a mass loss starting at 210° C., leading to LiCoO₂ and Co₃O₄ with further mass loss at 860° C. as Co₃O₄ decomposes to 3 CoO. The mass loss is depicted by the upper line labelled “TGA” in FIG. 3.

It can be concluded from the TGA data that [Li(ox)]₂[Co₅(OH)₈] decomposes on heating in air starting at 210° C., losing 31% mass in a single step. This can be explained by the decomposition of the material to LiCoO₂ and Co₃O₄ with a predicted mass loss of 29%:

[Li(ox)]₂[Co₅(OH)₈]→2 LiCoO₂+Co₃O₄+4H₂O+4 CO₂

Further heating leads to formation of CoO from Co₃O₄.

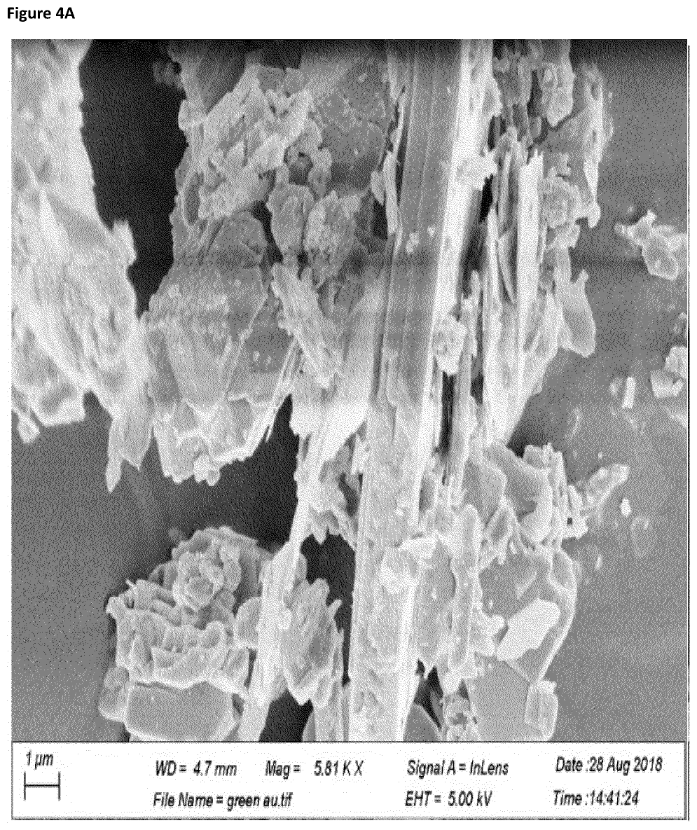

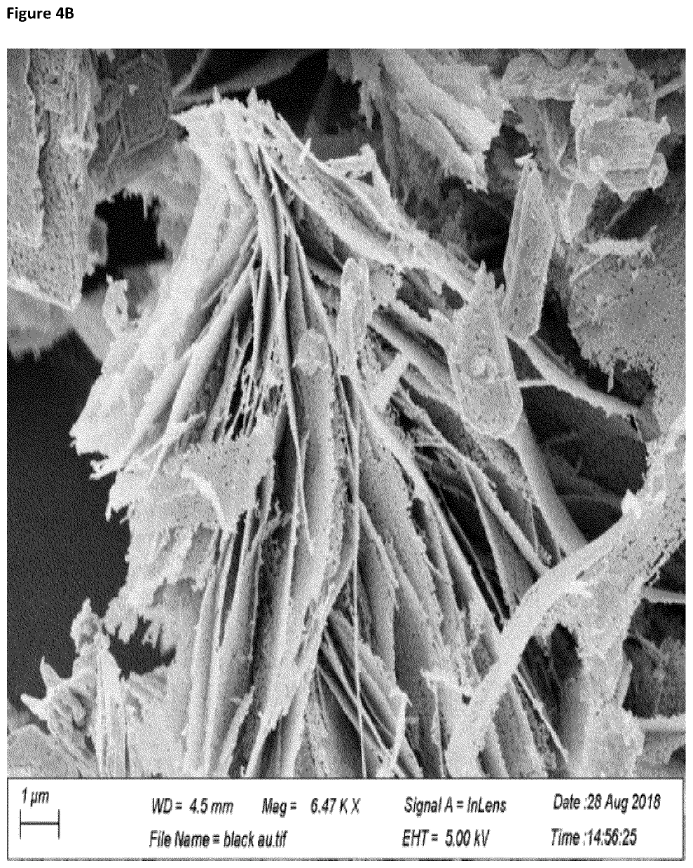

FIG. 4A and FIG. 4B show the SEM images of [Li(ox)]₂[Co₅(OH)₈] prior to and after decomposition. FIG. 4A indicates a side on view of plate like formation; FIG. 4B shows resultant of decomposition and delamination of the layered compound upon heating. This deflagration of the crystals into thin layers with grooves results from production of carbon dioxide and water vapour within the crystals upon decomposition to lithium metal oxide. Scanning electron microscopy (SEM) was carried out with Field Emission Zeiss Ultra Plus-SEM with GEMINI®FESEM column.

Referring now to FIG. 5 which shows the results of single crystal X-Ray diffraction of [Li(ox)]₂[M₅(OH)₈] where M=1:1:1: Ni:Mn:Co. Occupancy of each metal site fixed by electronic preference of metal(II) ions within the statistical mixture. Symmetry operators are the same as for FIG. 1.

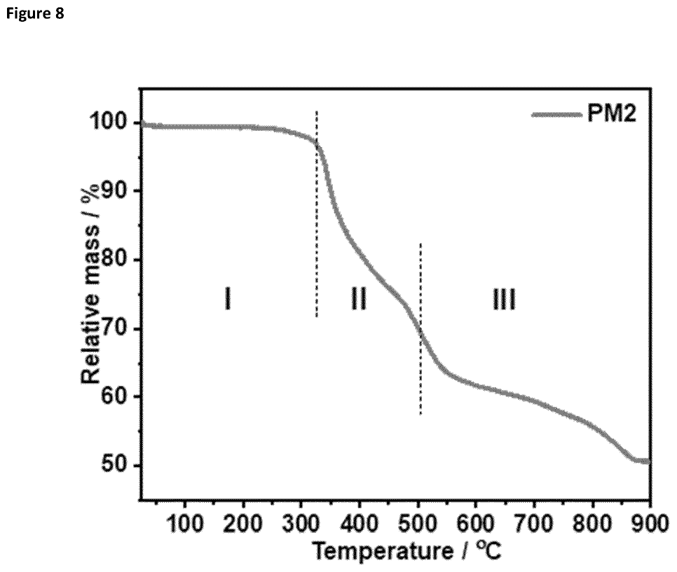

Since the crystal structure of [Li(ox)]₂[M₅(OH)₈] where M=1:1:1: Ni:Mn:Co shows the same layered configuration as the example where M=Co, it is primed to undergo the same decomposition upon heating, the results of which can be seen in FIG. 8.

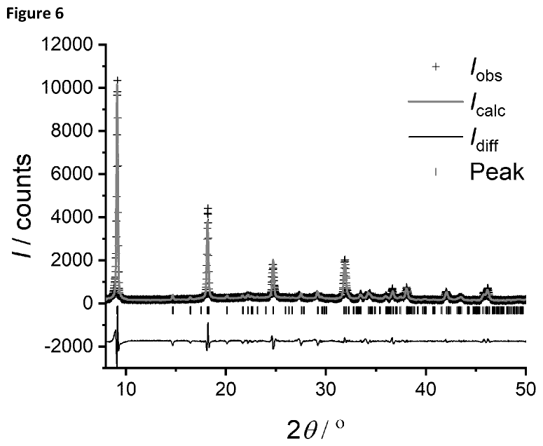

FIG. 6 shows the powder X-ray diffraction plot of [Li(ox)]₂[M₅(OH)₈] where M=1:1:1: Ni:Mn:Co.

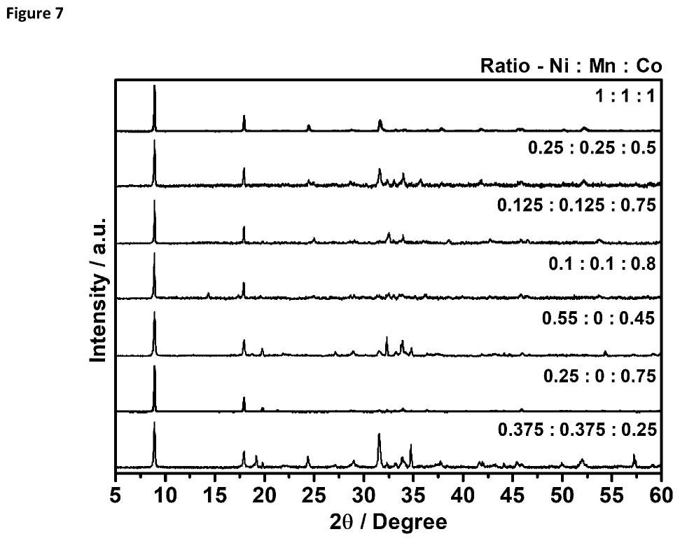

FIG. 7 shows the powder X-ray diffraction plot of [Li(ox)]₂[M₅(OH)₈] illustrating the wide range of ratios of Ni:Mn:Co that the compound of Formula I can comprise. The [Li(ox)]₂[M₅(OH)₈] was formed with the following ratios of Ni:Mn:Co—0:0:1, 1:1:1, 0.25:0.25:0.5, 0.125:0.125:0.75, 0.1:0.1:0.8, 0.55:0:0.45, 0.25:0:0.75, 0.375:0.375, 0.25. In some embodiments, the majority of metal present is nickel. In some embodiments, the majority of the metal is cobalt. In some embodiments no nickel is present. In some embodiments no manganese is present. In some embodiments, cobalt comprises the smallest fraction of all of the metals present in the compound of Formula I. The PXRD confirms that the phase is the same as for PM-1.

The above ratios which are only exemplary; the precursor forms over a wider range of ratios.

FIG. 8 shows that the decomposition of [Li(ox)]₂[M₅(OH)₈] where M=1:1:1: Ni:Mn:Co starts at 320° C. This is a higher temperature than that required for the decomposition wherein M=Co only. Range I—stable [Li(C₂O₄)]₂[M₅(OH)₈]; Range II: decomposition; Range III—LiMO₂+metal oxides where M=1:1:1 Ni:Mn:Co.

The invention is defined by the appended claims.

1. A compound of Formula I, [Li(ox)]₂[M¹ₘM²ₙM³ₖ(OH)ₚF_{q}];
wherein M¹, M² and M³ are metals; and
m, n and k are, independently, a number between 0 and 5, and the sum of m, n and k is 5; p and q are, independently, a number between 0 and 8, and the sum of p and q is 8.

2. The compound of claim 1 wherein M¹, M² and M³ are the same or different and are independently selected from cobalt, nickel and manganese.

3. The compound of claim 1 wherein M¹ is nickel, M² is manganese and M³ is cobalt; or wherein m and n are 0; k is 5; and M³ is cobalt.

4. The compound of Formula I of claim 1 wherein Formula I is [Li(ox)]₂[Co₅(OH)₈]; or
Formula I is [Li(ox)]₂[Ni_{5/3}Mn_{5/3}Co_{5/3}(OH)₈], wherein 5/3 represents five thirds.

5. A method for synthesizing the compound of claim 1, the method comprising: a first step comprising heating a first reaction mixture comprising a source of the metal or metals, M¹, M² and M³, a source of oxalate anions, lithium ions, and a base at a first reaction temperature for a first reaction time.

6. The method of claim 5, wherein the first heating step is carried out in a sealed reactor or an acid digestion vessel.

7. The method of claim 5, wherein one or more of M¹, M² and M³ is provided as a salt,
optionally wherein the, or each, M¹, M² and M³ salt is selected from a cobalt (II) or (III) salt, a manganese (II) or (III) salt or a nickel (II) or (III) salt, or a mixture thereof, or optionally wherein the, or each, M¹, M² and M³ salt is soluble in water under self-generated pressure, at a temperature of 90° C. up to 300° C. and a concentration of 0.5M up to 10M; or
optionally wherein the salt is a halide a carboxylate, an oxoanion or a mixture thereof.

8. The method of claim 5, wherein the source of metal or metals is selected from cobalt chloride or cobalt oxalate.

9. The method of claim 5, wherein the source of metal or metals and the source of oxalate anions are collectively selected from cobalt oxalate, manganese oxalate, nickel oxalate, cobalt oxalate or a mixture of two or more thereof, wherein the source of metal or metals and the source of oxalate anions is anhydrous or a hydrate.

10. The method of claim 5 wherein the source of oxalate anions is oxalic acid or an oxalate salt,
optionally wherein the oxalate salt is a dihydrate.

11. The method of claim 5, wherein the lithium ion is selected from a lithium salt or a lithium base or a mixture thereof, optionally, wherein the lithium base is lithium hydroxide,
optionally in anhydrous or monohydrate form, or
optionally wherein the anion of the lithium cation is a halide, a carboxylate, a halite, fluorosilicate, formate, molybdate, nitrate, nitrite, perchlorate, permanganate, selenide, selenite, sulfate, or thiocyanate, optionally wherein the source of fluorine is lithium fluoride.

12. The method of claim 5, further comprising the step of recovering the lithium ions from batteries or further comprising the step of recovering the source of the metal or metals from batteries,
optionally wherein the source of metal or metals is recovered by mixing the contents of a battery with an acid for at least 1 to 8 hours.

13. (canceled)

14. The method of claim 5, wherein the first reaction time is between 1 hour and 100 hours; and wherein the first reaction temperature is in the range from 90° C. to 400° C.

15. (canceled)

16. (canceled)

17. The method of claim 5, wherein halogen anions are not intentionally added to the source of the metal or metals, the source of oxalate anions, the lithium ions, and the base.

18. (canceled)

19. The method of claim 5, wherein the first heating step is carried out in a protic solvent, optionally water.

20. The method of claim 5 wherein, in the first heating step, the concentration of the metal or metals combined is at least 10 mmol/L, or
optionally, wherein the molar ratio of metal ions to lithium ions in the first reaction mixture before the first heating step is in the range of 1:10 to 5:1.

21. A method for synthesizing a lithium metal oxide, the method comprising:
forming a compound of Formula I by the method of claim 5, and further comprising a second heating step comprising heating the compound of Formula I at a second reaction temperature for a second reaction time to form the lithium metal oxide, wherein the compound of Formula I comprises:
[Li(ox)]₂[M¹ₘM²ₙM³ₖ(OH)ₚF_{q}];
wherein M¹, M² and M³ are metals; and
m, n and k are, independently, a number between 0 and 5, and the sum of m, n and k is 5;
p and q are, independently, a number between 0 and 8, and the sum of p and q is 8.

22. The method of claim 21, wherein the second reaction time is between 1 hour and 100 hours; and wherein the second reaction temperature is in the range from 200° C. to 900° C.

23. (canceled)

24. The method of claim 21 further comprising determining the second reaction temperature, optionally via thermogravimetric analysis of the compound of Formula I, or by heating the compound of Formula I and observing or detecting the temperature at which the reaction occurs.

25. The method of claim 21, wherein the second heating step is carried out in an open vessel or a laboratory flask.
